(19) United States
(12) Patent Application Publication
(10) Pub. No.: US 2005/0019237 A1
(43) Pub. Date: Jan. 27, 2005

(54) Sterilization tray assembly for medical instruments

(76) Inventor: Edward D. Riley, Falmouth, ME (US)

Correspondence Address: CESARI AND MCKENNA, LLP, 88 BLACK FALCON AVENUE, BOSTON, MA, 02210, US

(21) Appl. No.: 10/706,700

(22) Filed: Nov. 12, 2003

Related U.S. Application Data
(63) Continuation-in-part of application No. 10/675,186, filed on Sep. 30, 2003.
(60) Provisional application No. 60/488,892, filed on Jul. 21, 2003.

(51) Int. Cl. A61L 2/00

(52) U.S. Cl. 422/297; 422/292; 422/295; 422/300; 422/307

(57) ABSTRACT

A sterilization tray assembly for medical instruments has a unitary base including a plurality of tubes defining first passages, each first passage having open upper and lower ends, a first web connecting and supporting the tubes in parallel spaced-apart relation so that a fluid can circulate around and between the tubes, and instrument supports at the lower ends of the tubes for supporting medical instruments placed in the first passages while allowing a fluid to circulate through said first passages. The assembly also includes a unitary cover for seating on the base so as to cover and segregate the instruments supported by the base.

### CROSS-REFERENCE TO RELATED APPLICATION

This application is a continuation-in-part of Ser. No. 10/675,186, filed on Sep. 30, 2003, which claims the benefit of Provisional application No. 60/488,892, filed Jul. 21, 2003.

### BACKGROUND OF THE INVENTION

1. Field of the Invention

This invention relates to a sterilization tray assembly for holding selected sets of medical instruments or tools during sterilization and storage prior to use and for presenting them in a logical order when they are being used to perform a particular surgical procedure.

Different surgical procedures require the use of different sets of instruments or tools. For example, for a tooth implant, the instrument set may include twist drills, a drill extension, a driver, various size screws, etc. Prior to a particular surgical procedure, the surgeon determines or knows from experience which instruments will be required. The instruments are gathered together in a set, placed in a container which is packaged and then sterilized either chemically or in an autoclave. The packaged instrument set is then stored in that sterile condition until ready for use. When the surgeon is ready to perform the procedure, the container is brought to the operating room and opened exposing the still sterile instruments therein. At the end of the procedure, the instruments may be returned to the container and sent to a cleaning and sterilization facility.

2. Description of the Prior Art

The basic concept of organizing surgical instruments or tools into sets for subsequent sterilization, storage and presentation is not new; see, for example, U.S. Pat. Nos. 5,118,115 and 5,843,388. The former patent shows a perforated sterilization container assembly including a perforated housing with a removable cover and an internal rack or tray for supporting a set of medical instruments in an organized manner. More particularly, the instruments are vertically supported in grommets mounted to the tray at spaced-apart locations thereon. Graphics may be imprinted on the tray to identify the instruments at particular locations in the container.

While that assembly is satisfactory in many respects, it is disadvantaged in that it utilizes grommets for supporting the instruments. The grommets must be removed from the internal tray following surgery because blood and tissue tend to collect in the spaces between the grommets and the tray. This adds to the cost of cleaning and maintaining that container assembly.

The latter above patent discloses a sterilization rack for medical instruments and which has shelves containing cutouts for supporting medical instruments. The rack has open sides so that the instruments are not protectively enclosed within the rack. Also, the rack is composed of many different parts which must be assembled, adding to the cost of that device.

### SUMMARY OF THE INVENTION

Accordingly, the present invention aims to provide an improved sterilization tray assembly for sterilizing, storing and presenting a set of medical instruments.

Another object of the invention is to provide a sterilization tray assembly which protectively encloses the instruments.

A further object of the invention is to provide an assembly of this type which maintains the medical instruments at specified locations within the assembly.

Still another object of the invention is to provide such an assembly which is easy to clean and to sterilize.

Another object of the invention is to provide a sterilization tray assembly which is composed of a minimum number of parts which are relatively easy to manufacture.

Yet another object of the invention is to provide an assembly which is user friendly.

Other objects will, in part, be obvious and will, in part, appear hereinafter. The invention accordingly comprises the features of construction, combination of elements and arrangement of parts which will be exemplified in the following detailed description, and the scope of the invention will be indicated in the claims.

Briefly, my sterilization tray assembly comprises a unitary base having a top wall or web and a plurality of spaced-apart vertical tubes extending down from the top wall so as to define instrument-receiving passages having enlarged entrances at the top wall. The tubes have instrument supports partially occluding the lower ends of the passages for supporting instruments placed in the passages, while allowing a sterilant to circulate through those passages.

The assembly also includes a unitary cover for seating on the base so as to cover the instruments supported by the base. The cover has a top wall or web formed with a corresponding plurality of spaced-apart depending sleeves or tubes defining open-ended passages whose lower ends are of the same size as the entrances to the passages in the base. When the cover is seated on the base, corresponding ones of the tubes and sleeves are co-linear and capture the instruments therein, while still allowing the sterilant to contact the instruments.

Keying and latching means to be described later in detail facilitate the seating and locking of the cover on the base.

### BRIEF DESCRIPTION OF THE DRAWINGS

For a fuller understanding of the nature and objects of the invention, reference should be made to the following detailed description taken in connection with the accompanying drawings, in which:

FIG. 1 is a perspective view from above of a medical instrument sterilization tray assembly incorporating the invention;

FIG. 2 is a perspective view from above showing the base component of the FIG. 1 tray assembly;

FIG. 3 is a perspective view from below thereof;

FIG. 4 is a perspective view from below of the cover component of the FIG. 1 assembly, and

FIG. 5 is a cross-sectional view of the FIG. 1 assembly.

### DESCRIPTION OF THE PREFERED EMBODIMENT

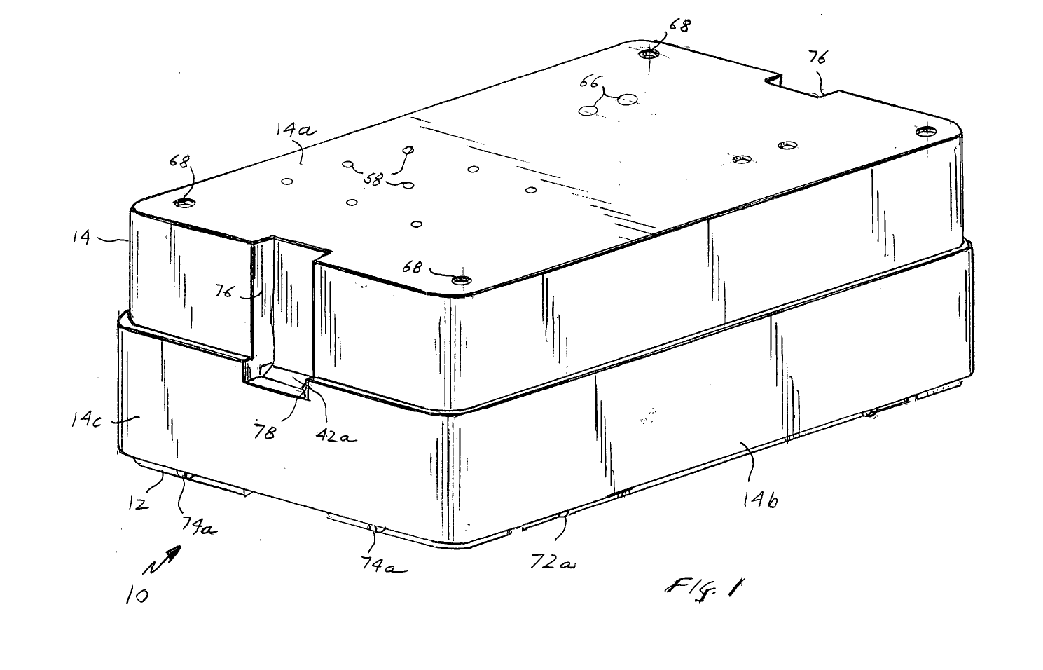

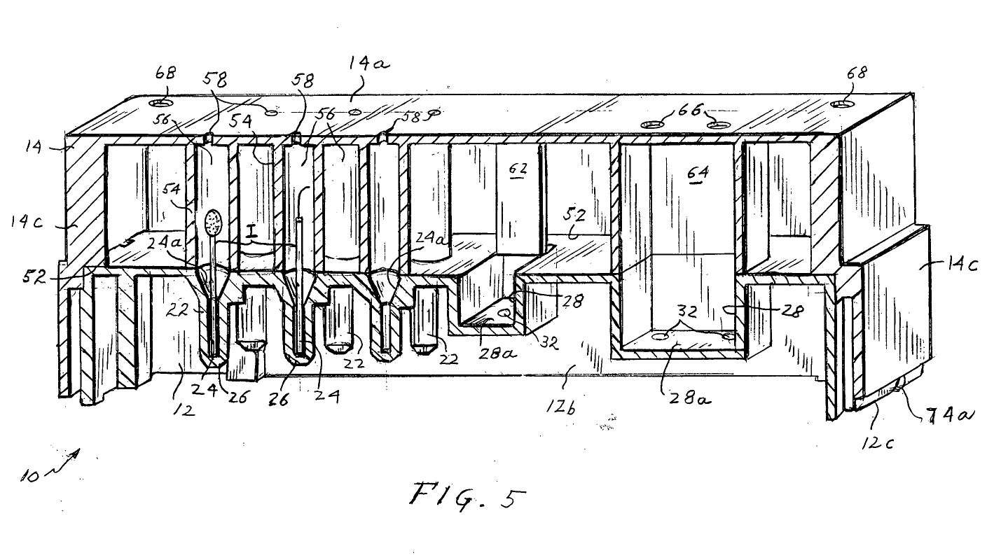

Referring to FIGS. 1 and 5 of the drawings, my tray assembly shown generally at 10 comprises a base 12 for supporting a plurality of medical instruments or tools I (FIG. 5) and a cover 14 which may be removably engaged to the top of base 12 to protectively enclose the instruments I (FIG. 5). Preferably, each of the base 12 and cover 14 is a unitary molded plastic part made of a rigid, impact resistant plastic material which is able to withstand sterilization, e.g. polyphenylsulphone. The dimensions of assembly 10 may vary depending upon the number of instruments contained in the assembly. The illustrated tray assembly suitable for holding a set of instruments for performing dental implants may be in the order 8 inches long, 4 inches wide and 2 inches high. The instrument set may include up to nine elongated drill bits or picks as well as other instruments or tools such as extenders, screws and the implants themselves.

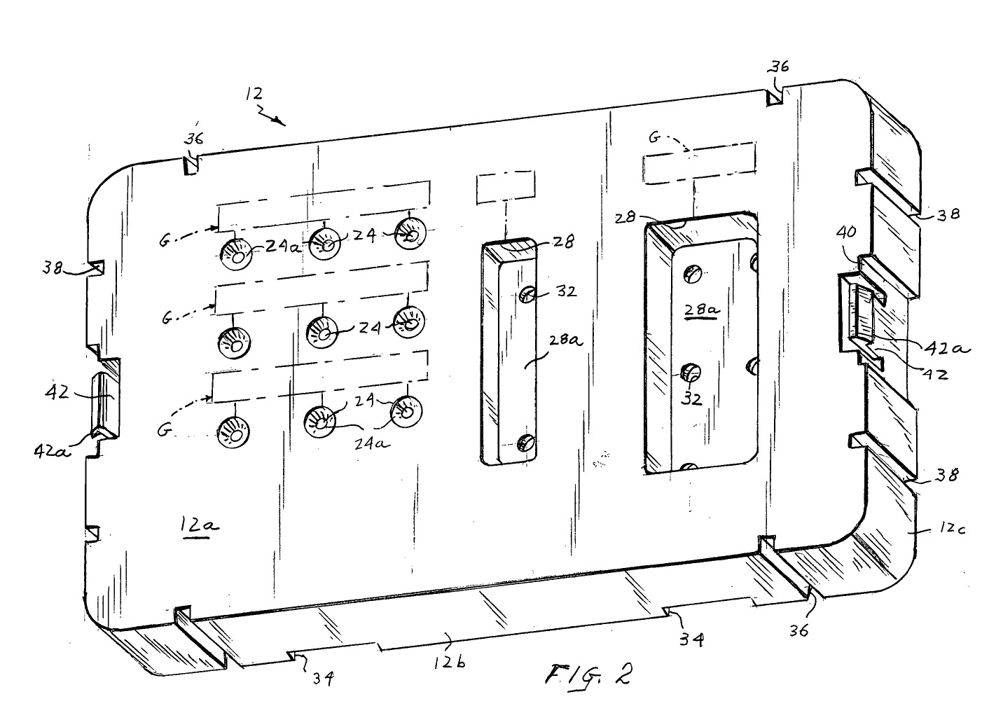

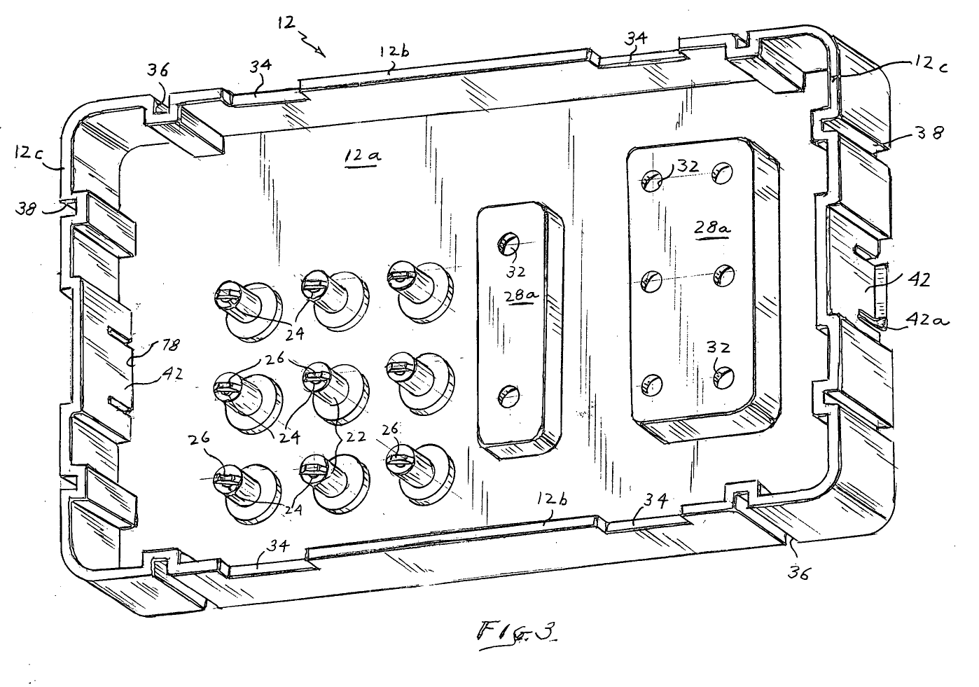

Referring now to FIGS. 2, 3 and 5, base 12 comprises a top wall or web 12a, a pair of opposite side walls or webs 12b, 12b and a pair of opposite end walls or webs 12c, 12c, all of the side and end walls extending down from top wall 12a. The bottom of base 12 is open as shown in FIG. 3.

Formed integrally with top wall 12a, is a plurality of spaced-apart tubes 22 which extend down from wall 12a so that a sterilant can circulate around the tubes. Preferably tubes 22 are arranged in columns and rows and they extend down a distance less than the heights of walls 12b, 12c. Each tube 22 has a lengthwise passage 24 with a flared or funnel-shaped entrance end 24a which opens at the top wall 12a of base 12. While the passages 24 are shown as being cylindrical, other shapes are possible, e.g. oval, rectilinear, etc.

The passage 24 in each tube extends all the way through the lower end of the tube. Also, the lower end of each tube 22 is formed with an integral bridge 26 which diametrically bridges the lower end of the passage 24 formed by that tube so that the bridge partially occludes the lower end of passage 24. Thus, when an instrument I is inserted into a passage 24 as shown in FIG. 5, the lower end of that instrument will be supported by the corresponding bridge 26. However, when cover 12 is secured to base 10 as shown in FIGS. 1 and 5, a sterilant is still able to flow into the lower end of each passage 24 on either side of the corresponding bridge 26. Alternatively, the passage 24 may be provided with reduced diameter lower ends to provide interior flanges that support instruments I.

In addition to the instrument-supporting tubes 22, the base top wall 12a may be formed with one or more receptacles 28. The illustrated receptacles are generally rectangular. However, they could just as well have other shapes and various dimensions. Receptacles 28 are adapted to contain instruments or tools which are not suitable for being supported in the passages 24, e.g. dental implants, fixtures, etc. Preferably, each receptacle 28 has a bottom wall 28a provided with one or more through holes 32 through which a sterilant may circulate into the corresponding receptacle 28.

Desirably, the side walls 12b and end walls 12c of base 12 extend down from top wall 12a so that the lower edges of the side and end walls are below the bridges 26 and the receptacle bottom walls 28a. Thus, when those lower edges rest on a supporting surface, the lower ends of passages 24 and the through holes 32 are spaced above that surface so that sterilant can be circulated around tubes 22 and through all of those passages. To optimize that circulation, the base walls 12b and 12c may be provided with one or more notches or through holes 34.

As shown in FIG. 2, if desired, graphics G may be printed on top wall 12a which identify the instruments contained in the various passages 24 and in the receptacles 28.

Referring to FIGS. 2 and 3, for reasons that will become apparent, the side walls 12b, 12b of base 12 may be provided with one or more vertical slots 36 which extend the full height of the base. Similar vertical slots 38 may be formed in the end walls 12c, 12c.

In order to releasably lock cover 14 to base 12 as shown in FIGS. 1 and 5, the opposite end walls 12c, 12c are notched at 40 and formed with a pair of upstanding resilient latches 42 which extend to the top of the base. Each latch 42 has an outwardly projecting nose 42a which can releasably engage cover 14 when the cover is seated on the base as shown in FIGS. 1 and 5.

Referring now to FIGS. 1, 4 and 5, cover 14 comprises a top wall or web 14a, a pair of opposite side walls or webs 14b, 14b and a pair of opposite end walls or webs 14c, 14c. The cover is formed with an interior overhang 52 which extends along the walls 14b, 14c part way up on the cover. When the cover 12 is seated on base 12 as shown in FIGS. 1 and 5, the overhang 52 rests on the perimeter of the base wall 12a to fix the elevation of the cover on the base.

Extending down from the cover top wall 14a is a plurality of sleeves 54 preferably arranged in columns and rows. The sleeves 54 define internal passage 56 whose cross-sections have the same shape and dimensions as those of the entry ends 24a of passages 24 in base 12. The sleeves 54 are arranged so that when the cover 14 is seated on base 10 as shown in FIGS. 1 and 5, the passages 56 in sleeves 54 are co-linear with passages 24 and aligned with corresponding passage ends 24a in base 10. As best seen in FIG. 5, the cross-sections of passages 56 are larger than those of passages 24 and the passages 56 are longer than passages 24 to accommodate the different heights and top shapes/sizes of the various instruments I that may be positioned in base 12.

Preferably, small through holes 58 are present in the cover top wall 14a in alignment with passages 56 so that when the cover is seated on the base, a sterilant can flow through the holes 58 into passages 56. These holes 58 should be smaller than the tops of instruments I so that the instruments will not fall through those holes if assembly 10 should be inverted.

As shown in FIG. 5, means are provided for holding in place the instruments or tools I contained in the receptacles 28 of base 12 when the cover is seated on the base. These holding means may take different forms. In the illustrated assembly 10, the cover, top wall 14a is formed with one or more depending tabs 62 which is/are located directly above the smaller receptacle 28 in base 10 when the cover is seated on the base. For holding the contents of the larger receptacle 28 in base 12, the cover wall 14a may be provided with a depending corral or fence 64 which, when cover 14 is placed on base 10, surrounds top opening into the larger receptacle 28 in base 12. Preferably, one or more through-holes 66 is/are provided in the cover top wall 14a at the location of corral 64 so that a sterilant can circulate within that space.

In the cover 14, the sleeves 54, tabs 62 and corral 64 extend down from the cover top wall 14a a distance no greater than the spacing of the overhang 52 from top wall 14a so that when the cover 14 is seated on base 12, the lower ends of those structures will be located at or close to the base top wall 12a as shown in FIG. 5.

Preferably, one or more additional through holes 68 are provided in the cover top wall 14a to optimize the circulation of sterilant within the cover when the cover is in place on the base 12.

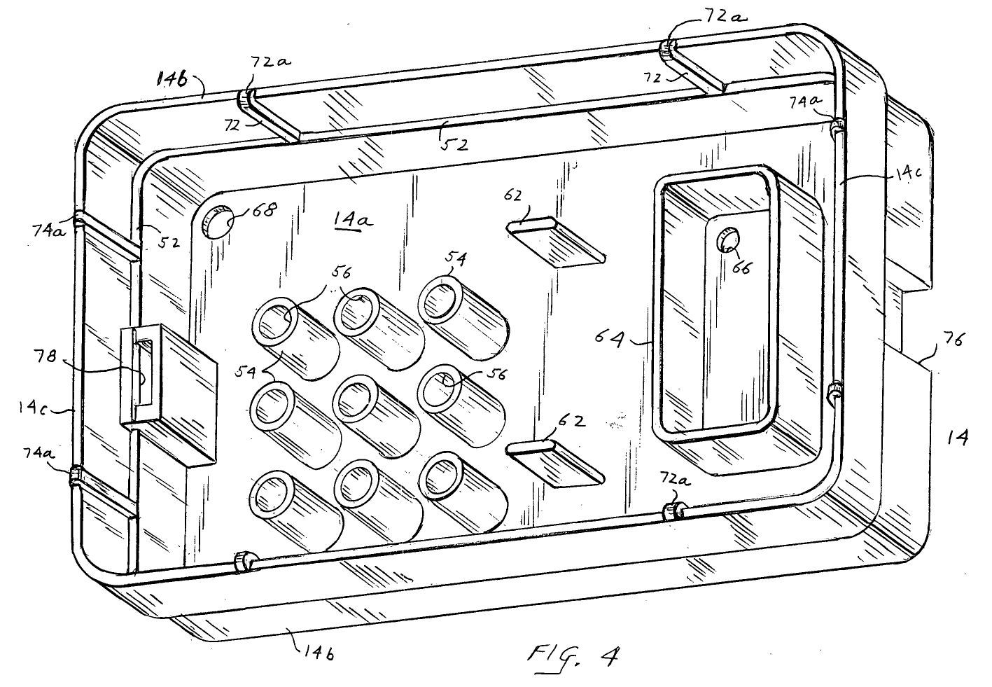

Referring now to FIGS. 4 and 5, to facilitate locating cover 14 on base 12, the cover side walls 14b, 14b are provided with one or more vertical interior keys or ribs 72 which extend along those walls below overhang 52. Each key or rib has a lower rounded end 72a which projects beyond the lower edge of the corresponding side wall 14b. The keys 72 are positioned along the side walls, 14b, 14b so that when the cover 14 is properly aligned with base 12 for seating thereon, the keys 72 will be aligned with the keyways 36 in the base. Similar interior keys 74 with lower projecting rounded ends 74a are formed on the cover end walls 14c, 14c, which keys are adapted to engage in the keyways 38 in base 10.

The mating keys and keyways have a relatively close sliding fit and they are distributed around the perimeter of assembly 10 so that cover 14 may be precisely aligned with base 12 in both the X and Y directions and brought into parallelism with the base when the keys are keyed into the corresponding keyways. This assures that the sleeves 54 in the cover are in precise axial alignment with the tubes 22 in base 10 so that the upper ends of the instruments I seated in tubes 22 of base 10 will pass easily into the sleeve passages 56 in the cover.

Preferably, the downwardly projecting ends 72a, 74a of keys 72, 74 are designed so that as the cover is seated on the base, they will engage in the keys 36, 38 in the base 10 to align the cover with the base as described above before the lower ends of the sleeves 54 pass over the upper ends of the instruments I supported in the passages 24 of base 10. This helps significantly in mating the cover 14 to base 12 with the instruments therein.

Of course, the positions of the keys and keyways may be reversed with the key ends 72a projecting up from the base for engagement in internal keyways in the cover 12.

In the illustrated assembly 10, the keys 72, 74 and keyways 36, 38 are located symmetrically in the assembly. However, it is preferable to locate those elements asymmetrically so that the cover can only be seated on the base one way, i.e. when the sleeves 54 are located over the passages 24 and the tabs 62 and corral 64 are positioned over the two receptacles 28.

As best seen in FIGS. 1 and 4, the end walls 14c, 14c of cover 14 are notched at 76 above the overhang 52 to accommodate lateral slots 78 at the opposite ends of cover 14. When the cover is seated on the base as shown in FIGS. 1 and 5, the latches 42 of the base are positioned to project through the slots 78 such that the latch noses 42a overhang the outer edges of the slots thereby locking the cover 14 to base 12. The cover may be released from the base by pressing in on the latches 42.

The tray assembly 10 may be packaged and sold as a stand alone assembly to individual physicians, clinics and offices where sets of medical instruments are used. In use, the assembly is removed from its sterile package (not shown) and the cover 14 is released from base 12 so that the instruments I held by the base are readily accessible to the user. The user simply selects instruments I from base 12, perhaps using the information provided by graphics G.

When the procedure is completed, the instruments may be returned to the base and the cover closed over the base allowing the instruments to be sent to a cleaning facility without fear of spreading contamination.

It will thus be seen that the objects set forth above, among those made apparent from the preceding description, are efficiently attained and, since certain changes may be made in the above construction without departing from the scope of the invention, it is intended that all matter contained in the above description or shown in the accompanying drawings shall be interpreted as illustrative and not in a limiting sense.

It is also to be understood that the following claims are intended to cover all of the generic and specific features of the invention described herein.

1. A sterilization tray assembly for medical instruments, said assembly comprising
a unitary base including
a plurality of tubes defining first passages, each first passage having open upper and lower ends,
a first web having a periphery and connecting and supporting said tubes in parallel spaced-apart relation so that a fluid can circulate around and between said tubes;
instrument supports at the lower ends of the plurality of tubes for supporting medical instruments placed in said first passages while allowing a fluid to circulate through said first passages, and
a unitary cover for seating on said base so as to cover said instruments.

2. The assembly defined in claim 1 wherein the upper ends of the first passages are flared.

3. The assembly defined in claim 1 wherein said base and said cover include inter-fitting latching surfaces which, when the cover is seated on the base, releasably secure the cover to the base.

4. The assembly defined in claim 1
wherein said first web constitutes a top wall of the base, and
further including a peripheral web extending down from said first web around said plurality of tubes, said peripheral web constituting a side wall of the base as well as defining a seating surface for the cover.

5. The assembly defined in claim 1 wherein said first web comprises a top web which extends between the upper ends of the tubes and constitutes a top wall of the base.

6. The assembly defined in claim 5 and further including one or more receptacles in said tray wall.

7. The assembly defined in claim 5 and further including graphics formed on said top wall and indicating the contents of said first passages.

8. The assembly defined in claim 1 wherein the instrument supports comprise bridges at the lower ends of the tubes which partially occlude the lower ends of the first passages.

9. The assembly defined in claim 1 wherein the instrument supports comprise interior flanges at the lower ends of said plurality of tubes.

10. The assembly defined in claim 1 wherein said cover includes
a plurality of sleeves defining second passages, each second passage having open upper and lower ends, said lower ends having substantially the same cross-sections as the upper ends of said first passages, and
a second web having a periphery and connecting and supporting the sleeves in parallel spaced-apart relation so that when the cover is seated on said base, the corresponding first and second passages are co-linear.

11. The assembly defined in claim 10 wherein the second passages are longer than the first passages.

12. The assembly defined in claim 10 wherein the upper ends of the second passages are smaller than the lower ends of the second passages.

13. The assembly defined in claim 12 wherein the cross-sections of the second passages are larger than those of the first passages.

14. The assembly defined in claim 10 and further including
first keying surfaces distributed around the periphery of the first web, and
second keying surfaces distributed around the periphery of the second web, said first and second keying surfaces being shaped and arranged to key together as the cover is seated on the base to bring said first and second passages into axial alignment.

15. The assembly defined in claim 14 wherein
the first web comprises the top wall of the base and the base also includes a side wall extending down from the top wall of the base,
the second web comprises the top wall of the cover and the cover also includes a side wall extending down from the top wall of the cover,
the first keying surfaces comprise exterior keyways in the side wall of the base which extend parallel to said tubes, and
the second keying surfaces comprise interior keys in the side wall of the cover which extend parallel to said sleeves, said keys being arranged to key into said keyways as the cover is seated on the base.

16. The assembly defined in claim 15 wherein the keys have projections extending below the bottom of the cover side wall so that said projections can be engaged in the keyways while the side wall of the cover is still spaced above the top wall of the base.

17. The assembly defined in claim 10 wherein the side wall of the base has a lower edge that defines a plane which is spaced below the lower ends of said tubes.

18. A sterilization tray assembly for medical instruments, said assembly comprising
a base including a top wall, a plurality of tubes extending down from said top wall in parallel spaced-apart relation, said tubes defining first passages, each first passage having an open upper end at said top wall and a lower end, instrument supports at the lower ends of the plurality of tubes for supporting medical instruments placed in said first passages while allowing a fluid to circulate through said first passages and a peripheral wall extending down from said top wall so as to surround said tubes, and
a cover for seating on the base so as to cover said instruments, said cover including a top wall, a plurality of sleeves extending down from said top wall of the cover in parallel spaced-apart relation, said sleeves defining second passages, each second passages having open upper and lower ends, said lower ends having substantially the same cross-sections as the upper ends of said first passages and a peripheral wall extending down from the top wall of the cover so as to surround said sleeves whereby when the cover is seated on the base, the corresponding first and second passages are co-linear and the peripheral wall of the cover overlaps the peripheral wall of the base.

19. The assembly defined in claim 18 and further including
first keying surfaces distributed around the peripheral wall of the base, and
second keying surfaces distributed around the peripheral wall of the cover, said first and second keying surfaces being shaped and arranged to key together as the cover is seated on the base.

20. The assembly defined in claim 19 wherein the first keying surfaces comprise exterior keyways extending perpendicular to the top wall of the base, and
the second keying surfaces comprise interior keys extending perpendicular to the top wall of the cover.

21. The assembly defined in claim 20 wherein said keys have projections which extend below the peripheral wall of the cover so that said projections can be engaged in said keyways while the peripheral wall of the cover is still spaced above the top wall of the base.
